(19) United States
(12) Patent Application Publication
(10) Pub. No.: US 2006/0199996 A1
(43) Pub. Date: Sep. 7, 2006

(54) Sling for supporting and occluding a tissue and method of using the same

(76) Inventors: Ricardo Caraballo, Hillsborough, NJ (US); Ian Asao Oyama, Maple Shade, NJ (US)

Correspondence Address: COZEN O'CONNOR, P.C., 1900 MARKET STREET, PHILADELPHIA, PA, 19103-3508, US

(21) Appl. No.: 11/073,048

(22) Filed: Mar. 4, 2005

(51) Int. Cl. A61F 2/00; A61F 13/00

(52) U.S. Cl. 600/37

(57) ABSTRACT

A method and sling for supporting a tissue or other lumen in a body. When the sling is used to support the urethra of a female, it is effective in treating urinary incontinence. In one embodiment, the invention is a sling comprising a first end portion and a second end portion; and a support section intermediate the first and second end portions for supporting a tissue (preferably a urethra), the support section having first and second occluding portions and a relief portion intermediate the first and second occluding portions. In another aspect, the invention is a method of using the sling to support and occlude a urethra.

### FIELD OF THE INVENTION

The present invention relates generally to the field of medical devices, and specifically to medical devices for supporting tissues.

### BACKGROUND OF THE INVENTION

Urinary incontinence arising from several conditions is a common symptom in many women, especially women who had previous vaginal deliveries. Stress urinary incontinence (“SUI”) is the involuntary loss of urine due to increases in intra-abdominal pressure associated with laughing, lifting, coughing, or other physical activity. SUI may be caused by excessive bladder neck mobility (hypermobility) and/or intrinsic sphincter deficiency (“ISD”). Bladder neck hypermobility is typically the result of weak periurethral and bladder support tissue which permits the movement of the bladder neck and proximal urethra during times of increased intra-abdominal pressure. ISD is an inherent weakness of the internal urinary sphincter due to scarring or denervation which renders the internal urinary sphincter incompetent. An incompetent urinary sphincter may allow SUI in the absence of bladder neck hypermobility as urine is pushed through the incompetent sphincter with increases in intra-abdominal pressure. Some patients have both bladder neck hypermobility and ISD resulting in extreme SUI.

A variety of techniques have arisen for treating SUI. The techniques primarily involve supporting the urethra in a position where the flow of urine may be controlled by urethral compression during increases in intra-abdominal pressure. FIG. 1 illustrates the problem. Internal parts 10 of a female include a bladder 12 and a urethra 14 leading from the bladder. The urethra is a relatively small tubular organ leading from the bladder to the external portion of the body. FIG. 1 also illustrates the pubic bone 18 and the vagina 16. The urethra is shown in a relatively unsupported position, slumped to the right in FIG. 1, where the urethral sphincter may be unable to control the flow of urine in the patient.

Prior art techniques include a variety of ways to support the urethra. These ways include suturing to musculature or fascia beneath the urethra. Perhaps the most popular recent methods have involved placing a sling or hammock beneath the urethra, and supporting the hammock by anchoring it to fascia or other suitable supports, such as rectus muscle, the pubic bone, Cooper's ligament, or to subcutaneous tissue above the rectus fascia. Some early prior art slings are depicted in FIGS. 2 and 3. In FIG. 2, a prior art sling 20 includes a central portion 22 and means for attaching 24, 26 on the ends of the sling. These means for attaching may include tabs as shown or may include a suture 28 to allow a surgeon to draw the ends of the sling through the patient. FIG. 3 depicts another prior art sling 30. This sling 30 has a central portion 32 with visual indicators 34 to aid the surgeon in positioning the sling under the urethra. The sling may be tapered towards the ends 36, and also has suture receiving sites 38 to resist tearing as the surgeon extends the sling through the body of the patient.

These prior art techniques have disadvantages in that they are not necessarily stable within the body of the patient. That is, once the sling is placed, it may tend to move, and thus the patient does not receive the benefit of the surgeon's precise placement of the sling for supporting the urethra and gaining the best control over incontinence. Other disadvantages lie in the design of the sling itself. Since at least the central portion of the sling has a constant width, it may be subject to rolling or bunching under the urethra. This may tend to re-form a wide band into a narrow supporting band underneath the urethra, providing less support and possibly cutting into the urethra in extreme cases.

In an effort to remedy these problems, newer sling designs have been introduced that include strain reliefs. Examples of such prior art slings are shown in United States Patent Application Publication 2004/0006353A1, Bosley, J R. et al., the teachings of which are herein incorporated by reference in its entirety. FIGS. 4 and 5 depict two embodiments of slings having strain relief designs. FIG. 4 depicts a prior art sling 40 having relief portions 45, 47. The relief portions 45, 47 are respectively located between the end portions 43, 44 and the support section 42. As a result the sling 40 will have a tend to bend more easily at the relief portions 45, 47, resulting in the sling 40 being able to be more easily curved into the proper position during implantation, and retain its curvature and orientation thereafter. FIG. 5, depicts a prior art sling 50 having strain reliefs in the form of serrations 51 along the length of the end portions 53, 54. The serrations 51 afford the end portions 53, 54 of the prior art sling 50 greater flexibility than the support section 50, resulting in the sing 50 being able to be more easily curved into the proper position during implantation, and retain its curvature and orientation thereafter. However, in both prior art slings 40, 50 the support sections 42, 52, which are positioned under the urethra of a patient, do not contain strain reliefs. As a result, the support sections 42, 52 resist bending/flexing. As will become apparent from the discussion of the present invention, this is an undesirable characteristic, resulting in the prior art slings 40, 50 performing a less than optimal job in prohibiting SUI.

### SUMMARY OF THE INVENTION

It is therefore an object of the present invention to provide a sling and method for supporting a urethra to treat SUI.

Another object of the present invention is to provide a sling and method for supporting a urethra that effectively occludes the urethra of a patient when intra-abdominal pressure is increased.

Yet another object of the present invention is to provide a sling and method for supporting a urethra that effectively occludes a greater length of the urethra of a patient than prior art slings and methods.

Still another object of the present invention is to provide a sling and method for supporting a urethra that can be used to treat SUI in conjunction with all existing implantation methods and procedures.

A further object of the present invention is to provide a sling and method for supporting a urethra that will reliably support the urethra, allowing a patient long-term relief from SUI.

It was discovered that when a patient having a modified sub-urethral tension free sling (“STS”) in position performed a valsalva maneuver, the STS appeared to occlude the urethra at the 3:00 and 9:00 positions. Efforts were then undertaken to design a sling that would take advantage of this discovery in order to achieve the objects set forth above.

The aforementioned objects are met by the present invention, which in one aspect is a sling for supporting and occluding a urethra comprising: a first end portion and a second end portion; and a support section intermediate the first and second end portions for supporting the urethra, the support section having first and second occluding portions and a relief portion intermediate the first and second occluding portions. By providing a relief portion on the support section of the sling that is located directly below the urethra when implanted, the sling will have a greater tendency to bend at the relief portion, thereby increasing the likelihood that the occluding portions will adequately contact and occlude the urethra at the 3:00 and 9:00 positions during a valsalva maneuver.

While it appears the inventors of the prior art slings disclosed in United States Patent Application Publication 2004/0006353A1 appreciated the value of strain reliefs to effectuate easier bending of prior art slings, the strain reliefs of prior art slings are positioned along the end portions or at the transition between the support section and the end portions. Thus, these prior art slings will have tendency to bend along the end portions rather than under the urethra (as the present invention), prohibiting proper occlusion of the urethra and decreasing the instances when the prior art sling operates effectively.

In some embodiments of the invention, the first and second occluding portions can be wider than the first and second end portions. Increasing the width of the occluding portions results in an increased length of the urethra being occluded during a valsalva maneuver. In one embodiment, the first and second occluding portions will have a width within a range of approximately 1 cm to 3 cm while the first and second end portions have a width of approximately 1 cm or less. The first and second occluding portions can take on a multitude of shapes, including without limitation, substantially rectangular, elliptical, semi-elliptical, trapezoidal, hexagonal, or triangular in shape. As used herein, the term ellipse includes a circle.

The relief portion can be formed by a variety of designs. In one embodiment, the relief portion is formed by at least one cutout, slit, or perforation. The cutout, slit, or perforation can take on any shape or orientation. In a preferred embodiment, the relief portion is formed by top and bottom cutouts. This results in the relief section being narrower than the first and second occluding portions, preferably within a range of approximately 0.5 to 1 cm. The top and bottom cutouts can be any shape, including without limitation, rectangular, semi-elliptical, or triangular in shape.

In another embodiment, the relief portion can be formed by a material that is thinner than the material of the first and second occluding portions. In still another embodiment, the relief portion can be formed by a material that is more flexible than the material of the first and second occluding portions.

In a preferred embodiment, the relief section can have a length within a range of approximately 1 cm to 2 cm. The sling can be constructed of any suitable materials, including without limitation a mesh material such as polypropylene, polyethylene terephthalate, and expanded polytetrafluoroethylene. Biological materials can also be employed for this purpose. A plastic sheath can be incorporated covering the sling if desired for ease of insertion into a patient. The sheath will be removed once the sling is properly implanted and positioned.

In another aspect, the invention is a method of supporting and/or occluding a urethra of a patient comprising: providing a sling comprising a first end portion, a second end portion, a support section intermediate the first and second end portions, the support section having first and second occluding portions and a relief portion intermediate the first and second occluding portions; and implanting the sling in the patient so that the relief portion is under a portion of the urethra.

When the sling is implanted according to the method of invention, when the patient performs a valsalva maneuver, the sling will bend at the relief portion, causing the first and second occluding portions of the sling to occlude urethra at the sides. Preferably, the occlusion occurs at the 3:00 and 9:00 positions.

When a sling is used that comprises a sheath covering, the implanting step will further comprise removing the sheath from the sling once the relief portion is under the portion of the urethra. Most preferably, at least approximately 2 cm of the urethra is occluded by the first and second occlusion portions. Any and/or all of the details discussed above with respect to the sling can be incorporated into the method of the invention if desired.

While the invention has been summarized with respect to the sling and method being used to support and/or occlude a urethra, it should be noted that the invention is not so limited and can be used to support any tissue.

### BRIEF DESCRIPTION OF THE DRAWINGS

FIG. 1 depicts a problem urethra requiring support.

FIG. 2 illustrates a first prior art sling for supporting a urethra.

FIG. 3 illustrates a second prior art sling for supporting a urethra.

FIG. 4 illustrates a third prior art sling for supporting a urethra having strain reliefs between the support portion and the end portions.

FIG. 5 illustrates a fourth prior art sling for supporting a urethra having strain reliefs on the end portions in the form of serrations.

FIG. 6 is a front view of a tension free sub-urethral sling (“STS”) according to a first embodiment of the present invention.

FIG. 7A is a schematic representation of the STS of FIG. 6 implanted in a patient according to an embodiment of the present invention while the patient is at rest.

FIG. 7B is a schematic representation of the STS of FIG. 6 implanted in a patient according to an embodiment of the present invention while the patient is performing a valsalva maneuver.

FIG. 8 is a front view of an STS according to a second embodiment of the present invention.

FIG. 9 is a front view of an STS according to a third embodiment of the present invention.

FIG. 10 is a front view of an STS according to a fourth embodiment of the present invention.

FIG. 11 is a front view of an STS according to a fifth embodiment of the present invention.

FIG. 12 is a front view of an STS according to a sixth embodiment of the present invention.

FIG. 13 is a front view of an STS according to a seventh embodiment of the present invention.

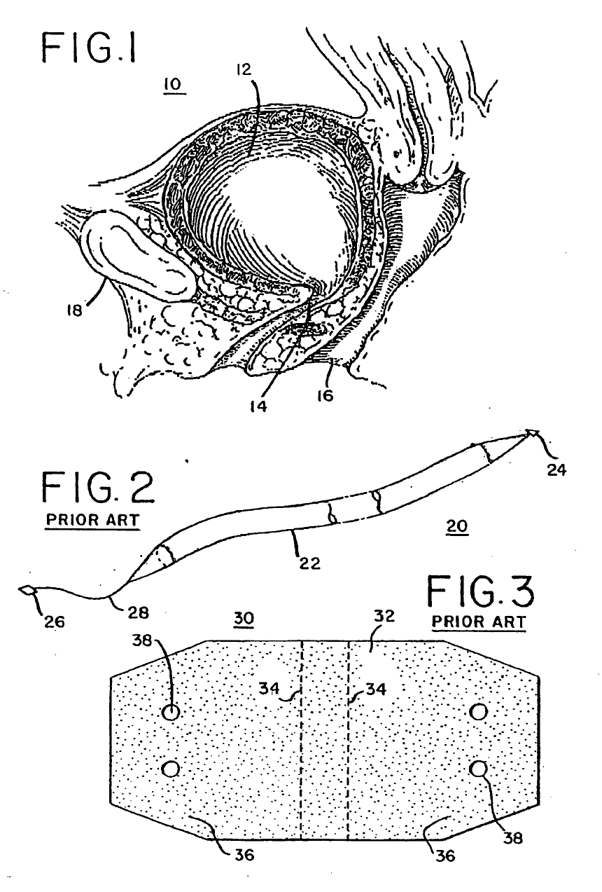

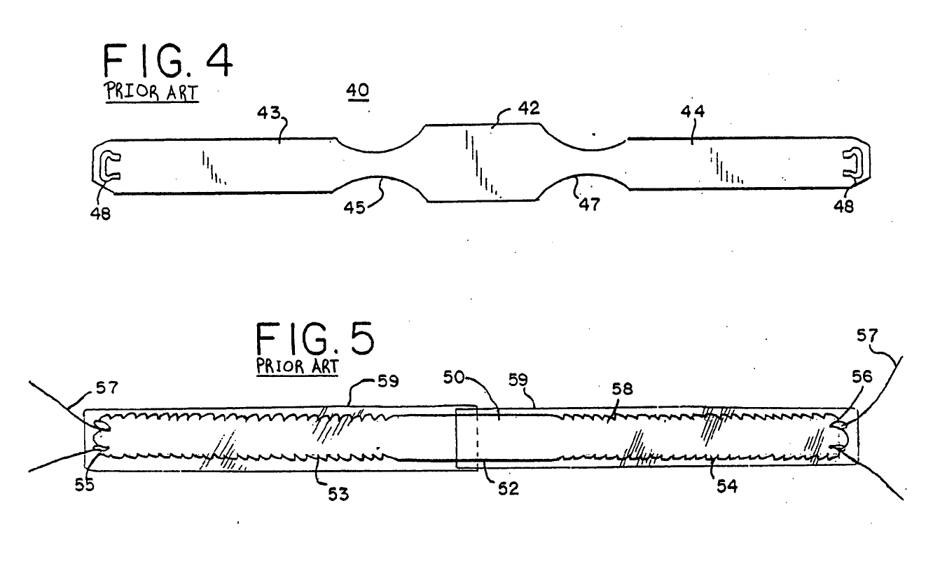

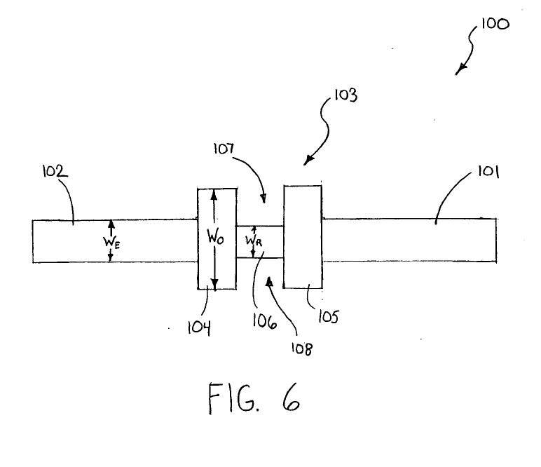

### DETAILED DESCRIPTION OF THE DRAWINGS

FIG. 6 is a front view of a tension free sub-urethral sling (“STS”) 100 according to one embodiment of the present invention. The STS 100 is constructed of a bio-compatible mesh material, such as a knitted polypropylene. The STS 100, however, is not limited to any specific material of construction and can be constructed of any materials, mesh or otherwise, that are suitable for sub-urethral sling construction. Examples of other materials include without limitation polypropylene, polyethylene terephthalate, and expanded polytetrafluoroethylene. Biological materials can also be employed for this purpose.

The STS 100 comprises two elongated end portions 101, 102 and a support portion 103. The support portion 103 is located intermediate of the two elongated end portions 101, 102 so that the two elongated end portions 101, 102 extend from opposite sides of the support section 103. The support section 103 comprises two occluding portions 104, 105 and a relief portion 106.

Depending on the desired characteristics, the entire STS 100 can be constructed of a single piece of material or the STS 100 can constructed of a plurality of separate pieces of material interwoven or otherwise joined together. For example, the entire support section 103 could be constructed from a different piece of material than the elongated end portions 101, 102 and then joined together. Or, the elongated end portions 101, 102 and the relief section 106 can be constructed of a single piece of material while the occluding portions 104, 105 are formed from separate pieces of material that are connect to the rest of the STS 100. In some embodiments, it may be desirable to form the occluding portions 104, 105 out of a more rigid material than the rest of the STS 100 so that the elongated end portions 101, 102 retain their flexibility while the occluding portion are more rigid so that they can more effectively occlude the urethra. In some embodiment, the elongated end portions 101, 102 may even be made of string or thread that is connected to the support section 103. The exact materials and design of construction of the STS 100 (and its components) will be dictated by costs considerations, a desired patient's anatomy, material availability, and FDA approval of materials and connection methods.

The occluding portions 104, 105 are used to contact and occlude the sides of the urethra of a patient. The occluding portions 104, 105 are rectangular in shape and are wider than the elongated end portions 101, 102. By designing the occluding portions 104, 105 to have a width W_{O} that is greater than the width W_{E} of the elongated end portions 101, 102, a greater length of a urethra will be occluded by the STS 100 while minimizing the intrusion of the STS 100 in a patient's body. It should be noted that in some embodiment, the occluding portions 104, 105 can be the same width of the elongated end portions 101, 102 if desired. Moreover, the occluding portions 104, 105 can take on an endless variety of shapes and still be within the scope of the present invention, including without limitation elliptical, semi-elliptical, trapezoidal, hexagonal, triangular, or irregular. Most preferably, forming the occluding portions 104, 105 out of shapes having sharp points is avoided to reduce the likelihood of damaging a patient's urethra during the application of occluding force.

In a preferred embodiment, the occluding portions 104, 105 will have a width W_{O} within a range of approximately 1 cm to 3 cm, and most preferably approximately 2 cm. The elongated end portions 101, 102 will preferably have a width WE within the range of 0.5 cm to 2.5 cm, and most preferably 1 cm.

The relief portion 106 is designed both: (1) to support the urethra of a patient; and (2) to provide an area of least resistance to bending of the STS 100. In STS 100, the relief portion 106 is formed by providing two cutouts 107, 108 near the center of the support section 106. As will be discussed below, there are numerous ways in which the relief portion can be formed. In the illustrated embodiment the cutouts 107, 108 are rectangular in shape. Those skilled in the art will understand that the shape of the cutouts 107, 108 is not limiting of the present invention and that the cutouts 107, 108 can take on any shape, including without limitation, elliptical, semi-elliptical, trapezoidal, hexagonal, triangular, or irregular. It is preferred that the cutouts 107, 108 be shaped so as not to have sharp points that can damage and cut the urethra.

In some embodiments, the relief portion 106 will have a width W_{R} that is less than the width W_{O} of the occluding portions 104, 105. In some embodiments, the width W_{R} of the relief portion 106 can even be less than the width W_{E} of the end portions 101, 102. Designing the relief portion 106 to have the smallest width of the component parts of the STS 100 helps ensure that the relief portion 106 has the least resistance to bending forces, thus, resulting in an implanted STS 100 bending at the relief portion 106 during a valsalva maneuver. The width W_{R} of the relief portion is preferably within the range of approximately 0.5 to 1 cm. However, the invention is not so limited, and in other embodiments, the width W_{R} of the relief portion 106 can be equal to or greater than the width W_{E} of the end portions 101, 102.

The length (measured left to right in FIG. 6) of the relief portion 106 is preferably sufficient to receive and support the urethra of a patient. Thus, the length of the relief portion 106 will depend on the patient, but will typically be in the range of 0.5 cm to 2 cm

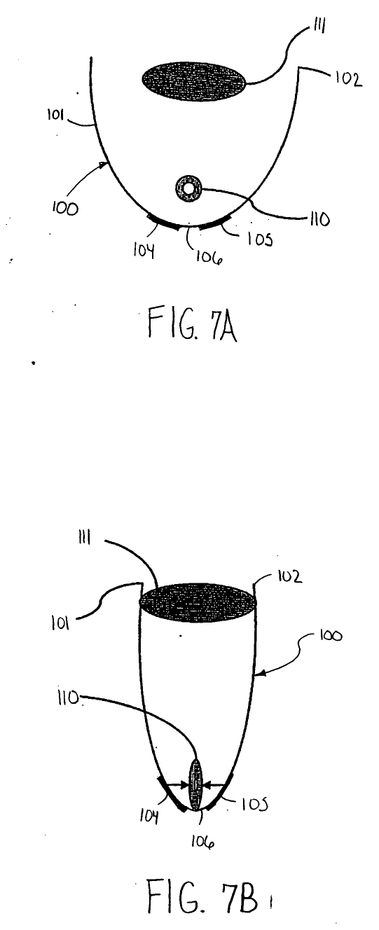

Referring now to FIGS. 7A and &B, a method of supporting and/or occluding the urethra 110 of a patient using the STS 100 will be discussed.

The STS 100 is first inserted into a patient using any well-established insertion methods/techniques and surgical instruments, until the STS 100 is oriented a shown in FIG. 7A.

For example, in using an older insertion method, the STS 100 can be positioned under the urethra 110 in a manner so that the relief portion 106 is below the urethra 10 and the two ends 101, 102 exit the patient's skin just above the pubic bone 111. With the patient laying on her back in the dorsal lithotomy position, an approximately 2 cm vertical incision is made in the vagina under the mid-portion of the urethra 110 (the average female urethra is 4-5 cm long). Through this incision, a small tunnel is created with scissors that will allow the STS 100 to lay under the urethra 110, as well as travel up its sides. An introducer needle is inserted into the vaginal incision, and into the right or left tunnel until the inferior aspect of the pubic bone 111 can be felt. The needle is directed around the pubic bone 111, and advanced until it exits the skin just above the pubic bone 111 (usually this ends up at the edge of the pubic hair line). Another introducer needle is used in a similar fashion, and placed along the opposite side of the urethra 110. (A typical introducer needle is curved and about 30 cm long). The STS 100 is attached to the ends of the needles in some fashion depending on the particular company that makes the needles you are using.

The needles are then pulled through the skin, bringing the STS 100 into place under the urethra 110 so that the relief portion 106 is below the urethra 110 (FIG. 7A), and the ends 101, 102 of the STS 100 extend through the skin (usually approximately 2-6 cm apart). In some embodiments, the STS 100 slings will have a protective sheath over it (not illustrated). Such sheaths are very well known in the art.

Once the STS 100 is in the position of FIG. 7A, the sheath is removed, exposing the edges of the mesh STS 100, which keeps the STS 100 fixed into place. The incision in the vagina is then closed with suture. The excess ends 101, 102 of the STS 100 that extend through the skin are trimmed and the puncture holes closed. The same procedure can be performed by starting the needle insertion at the skin, and directing it down toward the vaginal incision.

In a newer insertion method of placing the STS 100 under the urethra 110 is the trans-obturator technique. The STS 100 is the same, but the introducer needles have a different shape and the ends 101, 102 of the STS 100 exit the skin over the obturator foramen, and not the skin above the pubic bone 111. The obturator foramen is an opening in the pelvis that occurs where the leg attaches to the torso at the level of the clitoris. In a similar fashion to the original procedure with the patient in the dorsal lithotomy position, a 2 cm vertical incision is made in the vagina under the mid-portion of the urethra 110. Scissors are used to create a tunnel from this incision that travels under the urethra 110, and extends laterally toward the obturator foramen. An introducer needle pierces the skin over the obturator foramen, and is directed toward the vaginal incision as the needle curves around the bone 111 (outside-in technique).

The needles for the trans-obturator procedure are shorter, and can be curved or have a helical shape. The STS 100 is then attached to the needle, and the needle is brought back out of the pelvis delivering one of the ends 101, 102 of the STS 100 with it. This is repeated on the opposite side. The ends 101, 102 of the STS 100 are pulled into place, the protective sheath removed, and the incisions closed. Alternatively, an inside-out technique can be also be used.

Once the STS 100 is inserted as shown in FIG. 7A (and optionally the sheath removed if one was present), the STS 100 is in proper position to occlude the urethra 110 during a valsalva movement.

Referring to FIG. 7B, when the patient performs a valsalva maneuver, the urethral rotational descent results in the urethra applying pressure to the relief portion of the STS 100. As the force exerted from the urethral rotational descent continues, the STS 100 will bend at the relief portion 106, causing the occluding portions 104, 105 to contact and occlude the sides of the urethra 110 at the 3:00 and 9:00 positions. Because the occluding portions 104, 105 have an increased width, a greater length of the urethra 110 is occluded, increasing the likelihood of preventing SUI. The precise positioning of the STS 100, and the tendency of the STS 100 to bend at the relief portion 106 ensures that the urethra 110 is properly occluded.

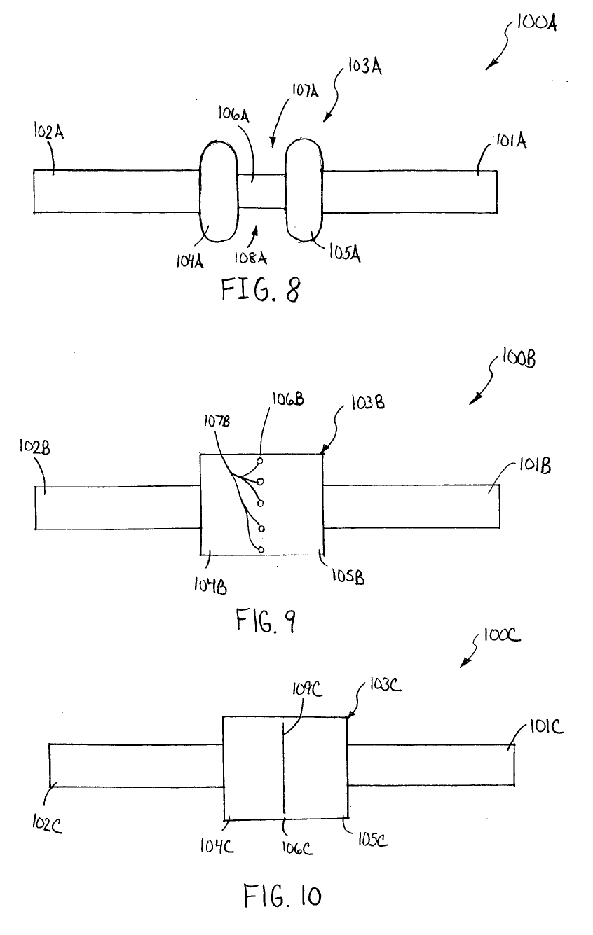

FIGS. 8-13 illustrate further embodiments of STSs according to the present invention. Any of the STSs can be used in the method described above. The discussion of these further embodiments will focus on those aspects that differ form STS 100 (namely construction of the relief portion) with the understanding that the details discussed above with respect to STS 100 are equally applicable. Like numbers are used to identify like parts with the exception of alphabetical suffixes being used for each embodiment.

Referring to FIG. 8, an STS 100A is illustrated. The STS 100A is identical to the STS 100 discussed above except that an alternative shape of the occluding portions 104A, 105A is implemented and the cutouts 107A, 108A are of a different shape.

Referring to FIG. 9, an STS 100B is illustrated. The STS 100B differs from STS 100 in that the relief portion 106B is formed by adding a plurality cutouts/perforations 107B to the center of the support section 103B between the occluding portions 104B, 105B.

Referring to FIG. 10, an STS 100C is illustrated. The STS 100C differs from STS 100 in that the relief portion 106C is formed by adding a slit 109C to the center of the support section 103C between the occluding portions 104C, 105C.

Referring to FIG. 11, an STS 100D is illustrated. The STS 100D is identical to the STS 100 discussed above except that a second alternative shape of the occluding portions 104D, 105D is implemented and the cutouts 107D, 108D are of a different shape.

Referring to FIG. 12, an STS 100E is illustrated. The STS 100E differs from STS 100 in that the relief portion 106E is formed by a material that is less resistive to bending forces than are the occluding portions 104E, 105E and the elongated end portions 101E, 102E. For example, if the STS 100E is constructed of polypropylene mesh, the relief portion 106E can be constructed so at to have larger thatching. In other embodiments, an entirely different material can be used to form the relief portion 106E than the rest of the STS 100E.

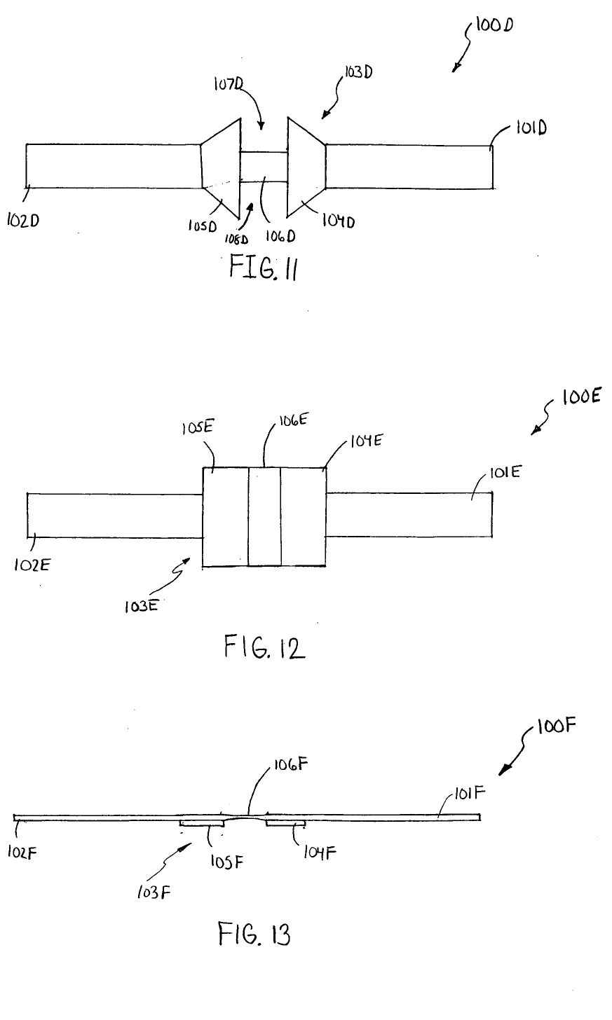

Referring to FIG. 13, a top view of an STS 100F is illustrated. The STS 100F differs from STS 100 in that the relief portion 106F is formed by thinning the material that forms the relief portion. As a result of this thinning, the relief portion 106F is less resistive to bending forces than are the occluding portions 104E, 105E and the elongated end portions 101E, 102E.

While the invention has been described and illustrated in sufficient detail that those skilled in this art can readily make and use it, various alternatives, modifications, and improvements should become readily apparent without departing from the spirit and scope of the invention. Specifically, the inventive sling and its method of use is not limited to supporting urethras and/or treating SUI. Those skilled in the art that the inventive sling and method can be used to support any tissue or lumen.

What is claimed is:

1. A sling for supporting and occluding a urethra comprising:
a first end portion and a second end portion; and
a support section intermediate the first and second end portions for supporting the urethra, the support section having first and second occluding portions and a relief portion intermediate the first and second occluding portions.

2. The sling of claim 1 wherein the first and second occluding portions are wider than the first and second end portions.

3. The sling of claim 2 wherein the first and second occluding portions are substantially rectangular, elliptical, semi-elliptical, trapezoidal, hexagonal, or triangular in shape.

4. The sling of claim 2 wherein the first and second occluding portions have a width within a range of approximately 1 cm to 3 cm.

5. The sling of claim 4 wherein the first and second end portions have a width of approximately 1 cm or less.

6. The sling of claim 1 wherein the relief portion is formed by at least one cutout, slit, or perforation.

7. The sling of claim 1 wherein the relief portion is formed by top and bottom cutouts, the relief section being narrower than the first and second occluding portions.

8. The sling of claim 7 wherein the relief portion has a width within a range of approximately 0.5 to 1 cm.

9. The sling of claim 7 wherein the top and bottom cutouts are rectangular, semi-elliptical, or triangular in shape.

10. The sling of claim 1 wherein the relief portion is formed by a material that is thinner than the material of the first and second occluding portions.

11. The sling of claim 1 wherein the relief portion is formed by a material that is more flexible than the material of the first and second occluding portions.

12. The sling of claim 1 wherein the relief section has a length within a range of approximately 0.5 cm to 2 cm.

13. The sling of claim 1 constructed of a synthetic or biological mesh material.

14. The sling of claim 1 further comprising a plastic sheath covering the sling.

15. The sling of claim 1 constructed of a mesh material; wherein the first and second occluding portions have a width that is greater than a width of the first and second end portions; wherein the relief portion comprises top and bottom cutouts that results in the relief portion having a width that is less than the width of the first and second occluding portions; wherein the first and second occluding portions has a width within a range of approximately 1 cm to 3 cm; wherein the first and second end portions have a width of approximately 1 cm or less; wherein the width of the relief portion is within a range of approximately 0.5 to 1 cm; wherein the relief section has a length within a range of approximately 0.5 cm to 2 cm.

16. A method of supporting and/or occluding a urethra of a patient comprising:
providing a sling comprising a first end portion, a second end portion, a support section intermediate the first and second end portions, the support section having first and second occluding portions and a relief portion intermediate the first and second occluding portions; and
implanting the sling in the patient so that the relief portion is under a portion of the urethra.

17. The method of claim 16 further comprising:
the patient performing a valsalva movement; and
in response to the valsalva movement, the sling bending at the relief portion causing the first and second occluding portions of the sling to occlude both sides of the urethra.

18. The method of claim 17 wherein the sling is covered with a sheath, the implanting step comprising removing the sheath from the sling once the relief portion is under the portion of the urethra.

19. The method of claim 17 wherein at least approximately 2 cm of the urethra is occluded by the first and second occlusion portions.

20. The method of claim 17 wherein the sling is constructed of a mesh material; wherein the first and second occluding portions have a width that is greater than a width of the first and second end portions; wherein the relief portion comprises top and bottom cutouts that results in the relief portion having a width that is less than the width of the first and second occluding portions; wherein the first and second occluding portions has a width within a range of approximately 1 cm to 3 cm; wherein the first and second end portions have a width of approximately 1 cm or less; wherein the width of the relief portion is within a range of approximately 0.5 to 1 cm; wherein the relief section has a length within a range of approximately 0.5 cm to 2 cm.
